(19) United States
(12) Patent Application Publication
(10) Pub. No.: US 2010/0280599 A1
(43) Pub. Date: Nov. 4, 2010

(54) CALCIUM PHOSPHATE COATED IMPLANTABLE MEDICAL DEVICES, AND ELECTROCHEMICAL DEPOSITION PROCESSES FOR MAKING SAME

(76) Inventors: Pui Hung Manus Tsui, Richmond (CA); Tomasz Trocynski, Vancouver (CA)

Correspondence Address: RISSMAN HENDRICKS & OLIVERIO, LLP, 100 Cambridge Street, Suite 2101, BOSTON, MA, 02114, US

(21) Appl. No.: 12/305,867

(22) PCT Filed: Jun. 20, 2007
(86) PCT No.: PCT/CA07/01100

§ 371 Date: Jun. 22, 2010

Related U.S. Application Data
(60) Provisional application No. 60/815,259, filed on Jun. 21, 2006.

(51) Int. Cl. A61L 27/32; A61F 2/02; C25D 9/00; C25D 7/00; C23C 28/00; A61L 27/54

(52) U.S. Cl. 623/1.42; 205/209; 205/210; 205/50; 205/198; 623/1.46

(57) ABSTRACT

This invention relates to novel calcium phosphate coated implantable medical devices, and electrochemical deposition processes for making same. A process of coating an implantable medical device with a calcium phosphate coating comprising: (a) subjecting a substrate to a surface pretreatment whereby adhesion of the calcium phosphate coating to the substrate is enhanced; (b) immersing the pretreated substrate in an electrolyte comprising calcium and phosphate species; and (c) coating calcium phosphate onto the substrate by electrochemical deposition.

### FIELD OF THE INVENTION

This invention relates to novel calcium phosphate coated implantable medical devices, and electrochemical deposition processes for making same.

### BACKGROUND OF THE INVENTION

Coronary artery disease (CAD) occurs when fat deposits block the arteries, reducing the oxygen supply to the heart muscle. Angioplasty is a way of opening a narrowed or closed blood vessel without having to do major surgery. Between 70 percent and 90 percent of angioplasty procedures use a stent, a hollow thin-walled wire mesh tube, to keep the vessel open after widening. The stent is placed onto a balloon and pressed firmly against the artery wall during inflation. The balloon is then deflated, leaving the stent in place to act as a scaffold.

Metallic stents have been used by cardiologists to battle CAD with some success. Though the stent is an effective solution in providing structural support it does not eliminate the recurrence of blockage in the artery (restenosis) in all cases. The release of ions from the bare metal stent may result in the proliferation of smooth muscle cells, a natural inflammatory response to this foreign body. Narrowing or re-closing of the artery often requires a repeat operation within a year. An approach to address this issue is to coat the stent with a polymer coating that contains a drug that prevents the restenosis. A problem with this approach relate to lack of proper biocompatibility of the polymers (many of them trigger inflammatory response of the tissue). This may become an even more severe issue when the drug is entirely released from the coating.

Due to the excellent biocompatibility of calcium phosphate ceramics, they have received much attention in the fields of orthopedics and dentistry and are clinically applied. Among the various forms of calcium phosphate ceramics, hydroxyapatite [Ca₁₀(PO₄)₆(OH)₂] (HAP), a major inorganic component of natural bone, is particularly attractive. HAP, in the form of coatings on rigid implants such as orthopedic and dental implants, have been applied to achieve biocompatibility. Due to the brittle nature of calcium phosphates, there exists a challenge for calcium phosphates coatings to withstand implant deformation, such as stent expansion during the angioplasty procedure.

### SUMMARY OF THE INVENTION

One aspect of the present invention relates to novel calcium phosphate coated implantable medical devices, and electrochemical deposition processes for making same. A process of coating an implantable medical device with a calcium phosphate coating comprising: (a) subjecting a substrate to a surface pretreatment whereby adhesion of the calcium phosphate coating to the substrate is enhanced; (b) immersing the pretreated substrate in an electrolyte comprising calcium and phosphate species; and (c) coating calcium phosphate onto the substrate by electrochemical deposition.

Another aspect of the invention relates to a process of coating an implantable medical device with a composite ceramic/polymer coating comprising: (a) subjecting a substrate to a surface pretreatment whereby adhesion of the calcium phosphate coating to the substrate is enhanced; (b) immersing the pretreated substrate in an electrolyte comprising calcium, phosphate and polymer species; and (c) coating a continuous calcium phosphate phase and a continuous polymer phase onto the substrate by electrochemical deposition.

Yet another aspect of the invention relates to a process of coating an implantable medical device with a composite ceramic/polymer coating comprising: (a) subjecting a substrate to a surface pretreatment whereby adhesion of the calcium phosphate coating to the substrate is enhanced; (b) immersing the pretreated substrate in an electrolyte comprising calcium and phosphate species; (c) coating a continuous calcium phosphate phase onto the substrate by electrochemical deposition; and (d) impregnating the calcium phosphate coated substrate with a polymer to provide a continuous polymer phase.

A further aspect of the invention relates to an implantable medical device comprising a substrate with a surface micro-etched by alkaline pre-treatment, and a calcium phosphate coating deposited on the substrate by electrochemical deposition.

A still further aspect of the invention relates to an implantable medical device comprising a substrate with an oxidized surface layer; and a calcium phosphate coating deposited on the substrate by electrochemical deposition.

Another aspect of the invention relates to an implantable medical device comprising a substrate with aero-sol-gel deposited hydroxyapatite layer, and a calcium phosphate coating deposited on the substrate by electrochemical deposition

Yet another aspect of the invention relates to an implantable medical device comprising a substrate with a composite coating, the composite coating comprising a continuous ceramic phase and a continuous polymer phase.

A further aspect of the invention relates to the use of a coated implantable medical device comprising a continuous ceramic phase and a continuous polymer phase in an engineered drug delivery system.

### DRAWINGS

Exemplary embodiments are illustrated in referenced figures of the drawings. It is intended that the embodiments and figures disclosed herein are to be considered illustrative rather than restrictive.

FIGS. 1(a) to (d) illustrate an ultrasonically cleaned, unmodified stent made of 316L stainless steel. FIGS. 1(a) to (c) are micrographs of the stent and FIG. 1(d) is the stent viewed under an optical microscope.

FIG. 2 is a schematic diagram of an experimental setup for electrochemical deposition of HAP, with relevant chemical reactions, according to one embodiment of the present invention.

FIG. 3 is an X-ray diffraction graph illustrating the composition of electrochemically deposited calcium phosphate.

FIGS. 4(a) to (d) are micrographs illustrating an electrochemically deposited HAP coating on a stent according to Example 1.

FIGS. 5(a) to (d) are micrographs illustrating an electrochemically deposited HAP coating on a stent according to Example 1.

FIGS. 6(a) and (b) are micrographs illustrating an expanded HAP coated stent according to Example 1.

FIG. 7(a) is a micrograph illustrating an expanded HAP coated stent according to Example 1 showing details of coating damage in the compressive stress area.

FIG. 7(b) is a micrograph illustrating an expanded HAP coated stent according to Example 1 showing details of coating damage in the tensile stress area.

FIGS. 8(a) and (b) are micrographs illustrating an oxidation pretreated stent surface according to Example 2.

FIGS. 9(a) to (d) are micrographs illustrating electrochemically deposited HAP on an oxidation pretreated stent according to Example 2.

FIGS. 10(a) to (d) are micrographs illustrating an expanded HAP coated stent with oxidation pretreatment according to Example 2.

FIGS. 11(a) and (b) are micrographs illustrating an alkaline pretreated stent surface according to Example 3.

FIGS. 12(a) to (d) are micrographs illustrating electrochemically deposited HAP on an alkaline pretreated stent according to Example 3.

FIGS. 13(a) to (d) are micrographs illustrating an expanded HAP coated stent with alkaline pretreatment according to Example 3.

FIGS. 14(a) to (d) are micrographs illustrating an expanded HAP coated stent with alkaline pretreatment according to Example 4.

FIGS. 15(a) to (d) are micrographs illustrating an expanded HAP coated stent with alkaline pretreatment according to Example 4.

FIGS. 16(a) to (d) are micrographs illustrating an expanded HAP coated stent with alkaline pretreatment according to Example 4.

FIGS. 17(a) to (d) are micrographs illustrating an expanded HAP coated stent with alkaline pretreatment, coated in 60 seconds with a 0.75 μm coat, according to Example 5.

FIGS. 18(a) to (d) are micrographs illustrating an expanded HAP coated stent with alkaline pretreatment, coated in 70 seconds with a 0.90 μm coat, according to Example 5.

FIGS. 19(a) to (d) are micrographs illustrating an expanded HAP coated stent with alkaline pretreatment, coated in 90 seconds with a 1.0 μm coat, according to Example 5.

FIG. 19(e) are micrographs illustrating changes in thickness and density of the HAP coating on expanded stents for 40, 50, 60, 70 and 90 seconds of deposition, according to Example 5.

FIGS. 20(a) to (d) are micrographs illustrating a stent coated with 0.2 μm aero-sol-gel deposited film of HAP, according to Example 6.

FIGS. 21(a) to (d) are micrographs illustrating a stent coated with 0.2 μm aero-sol-gel deposited film of HAP and further coated with 0.5 μm HAP by ECD, according to Example 6.

FIGS. 22(a) to (d) are micrographs illustrating an expanded HAP stent coated with 0.2 μm aero-sol-gel deposited film of HAP and further coated with 0.5 μm HAP by ECD, according to Example 6.

FIGS. 23(a) to (d) are micrographs illustrating a stent coated by co-deposition of HAP and PVA (of 0.1 g in 80 ml of the coating solution), according to Example 7.

FIGS. 24(a) to (d) are micrographs illustrating an expanded stent coated by co-deposition of HAP and PVA (of 0.1 g in 80 ml of the coating solution), according to Example 7.

FIGS. 25(a) to (d) are micrographs illustrating a stent coated by co-deposition of HAP and PVA (of 0.5 g in 80 ml of the coating solution), according to Example 7.

FIGS. 26(a) to (d) are micrographs illustrating an expanded stent coated by co-deposition of HAP and PVA (of 0.5 g in 80 ml of the coating solution), according to Example 7.

FIGS. 27(a) to (d) are micrographs illustrating an expanded stent coated with HAP and impregnated with PLGA, according to Example 8.

FIGS. 28(a) to (d) are micrographs illustrating an expanded stent coated with HAP and impregnated with PLGA, according to Example 8.

FIGS. 29(a) to (d) are micrographs illustrating the surface morphologies of HAP coated stents impregnated with PLGA solutions at 2, 4 and 6 wt % concentrations, according to Example 8.

### DETAILED DESCRIPTION OF THE INVENTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

In the following description, the term “calcium phosphate” is used generically and includes minerals such as HAP, dicalcium phosphate, tricalcium phosphate, tetracalcium phosphate and amorphous or partially amorphous calcium phosphate.

The present invention in one aspect relates a process of coating an implantable medical device with a calcium phosphate coating comprising subjecting a substrate to a surface pretreatment whereby adhesion of the calcium phosphate coating to the substrate is enhanced, immersing the pretreated substrate in an electrolyte comprising calcium and phosphate species, and coating calcium phosphate onto the substrate by electrochemical deposition.

The novel coating process is exemplified below with reference to stents, such as cardiovascular stents (e.g. coronary stents). As shown in the examples below, the coating withstands simulated stent expansion procedures. However, the invention has broad application to virtually any type of implantable device with a metallic surface for use in the human or animal body, and particularly to flexible implantable devices that undergo substantial deformation during use. For example, the coatings are also useful in ureteral stenting and catherterisation.

The coatings are distinguished by a uniform and optimum thickness (<1 μm), and the coating adhesion is high enough to avoid separation of the coating from the substrate during implantation and expansion of the stent. The coatings are porous, typically in the range of 30-60 vol % porosity. The open porosity of the coatings may be filled with secondary materials such as polymers, proteins, drugs, and others.

Electrochemical deposition (ECD) is accomplished by using an electrolyte that contains calcium phosphate precursors and by application of a current that triggers precipitation of the calcium phosphate of a desirable phase (e.g. HAP) on one of the electrodes. The substrate deposition is preceded by surface pretreatment. As a result of the surface pretreatment, there is a surprising and significant increase in adhesion of the calcium phosphate coating to stents.

The increase in adhesion is believed to take place due to (i) surface nano-roughness created during pretreatment which enhances the mechanical interlocking of the hydroxyapatite coating; and/or (ii) a change of surface chemistry that promotes formation of chemical bonds between the modified surface and the coating during the ECD process. Surface modification is achieved by using (a) alkaline (micro-etching) treatment, (b) pre-oxidation treatment of the metallic stent surface or (c) pre-coating of the stent surface with sol-gel derived HAP. A combination of these surface modification methods may also be practiced.

During alkaline treatment, a nano-rough surface microstructure forms on the substrate, which is believed to promote mechanical interlocking and thus physical bonding. Additionally during alkaline treatment, an interfacial compound of Na₄CrO₄ forms, which may act as a chemical bonding bridge between metal and ceramic.

Specifically, pretreated 316L stainless steel stent substrates (using the above surface treatment methods (a), (b) and (c); refer to the examples below for the specifics) are subsequently coated by ECD using a specially designed electrolysis cell. The electrochemical deposition of calcium phosphates, e.g. hydroxyapatite, on the substrates are conducted in a mixed aqueous solution of calcium and phosphate species, such as Ca(NO₃)₂.4H₂O and NH₄H₂PO₄. The stainless steel stent substrate is the cathode (negative electrode), and a nickel ring, for example, is used as the anode (positive electrode). The pH of the electrolyte may be controlled at pH=4.0±0.1 with dynamic addition of suitable amount sodium hydroxide to the solution. The environment temperature may be controlled at T=50° C.±1° C.

Substrates are coated using specified parameters of current and duration of deposition. The specific parameters depend on the overall surface area of the substrate, and the desired thickness of the coating. For example, in one frequently used process with stents we have used a 0.90 mA applied current, and a deposition time of 50 seconds. The total current is specific to assure suitable current density of the stent surface. The deposition time is adjusted to achieve desired thickness of the deposit, i.e. about 1 um for the 50 second deposition for the above conditions. The combination of current, time, and electrolyte parameters control the coating uniformity, porosity, thickness, and phase composition. After achieving the desired coating thickness, the coated stents are rinsed in distilled water and dried.

The present invention in another aspect relates to improvements to the functional properties and reliability of implantable medical device coatings impregnated with polymers, or polymers containing drugs, for long-term controlled drug release. Porous calcium phosphate coatings (e.g. HAP coatings) can be used as a scaffold for carrying organic material, forming a novel organo-ceramic composite. A unique and distinctive feature of such a scaffold carrying the organic material is the physical continuity of the scaffold. In this regard, the present invention provides for implantable medical devices, and process for making same, wherein calcium phosphate coatings (i.e. the physically continuous scaffold) are combined with organic material (e.g. bio-polymers) either through co-deposition, or post-deposition impregnation.

Mechanical performance of the coated stents are tested by the stent expansion test. The expansion test determines the effect of surface treatment on damage to the HAP coating. During the test, stents expand to about three times their original diameter of about 1 mm. All expansion tests are performed with the commercial Encore™ 26 Inflation Device Kit. The catheter pressure used for expansion is 170 psi. After expansion, the adhesion of the HAP coating is investigated. Surface morphology and elemental analysis of the deposited specimens are observed by a Hitachi S-3000 scanning electron microscopy (SEM), with magnification of up to 10,000×.

We have observed that the calcium phosphate coatings according to the present invention are able to survive large strain (>10%) of the underlying substrate. Tiny fractures observed in some such coatings to accommodate the strain is distributed and localized, i.e. the nano-cracks are limited to small (<100 nm) areas adjacent to the pores. Although frequently not visible even under 10,000× magnification, collective opening of these nano-cracks accommodates the substrate strain. These nano-cracks may link to form larger (visible), 1-10 μm long cracks, but generally do not cause macro-cracking (>100 μm) leading to separation of the coating from the substrate. Denser (porosity <40%) and thicker (>1 μm) calcium phosphate coatings show macro-cracking during stent deformation. These kinds of calcium phosphate coatings are suitable for non-deforming substrates. However, these denser and thicker films are still suitable for deforming substrates (e.g. stents) if combined with viscoelastically deforming filler such as organic polymers.

### EXAMPLES

To demonstrate the feasibility of the novel processing concepts outlined above, the following examples are described below for a stainless steel substrate, in particular, coronary stents. The procedures outlined below can be applied to other implantable medical devices.

### Example 1

### General ECD Process

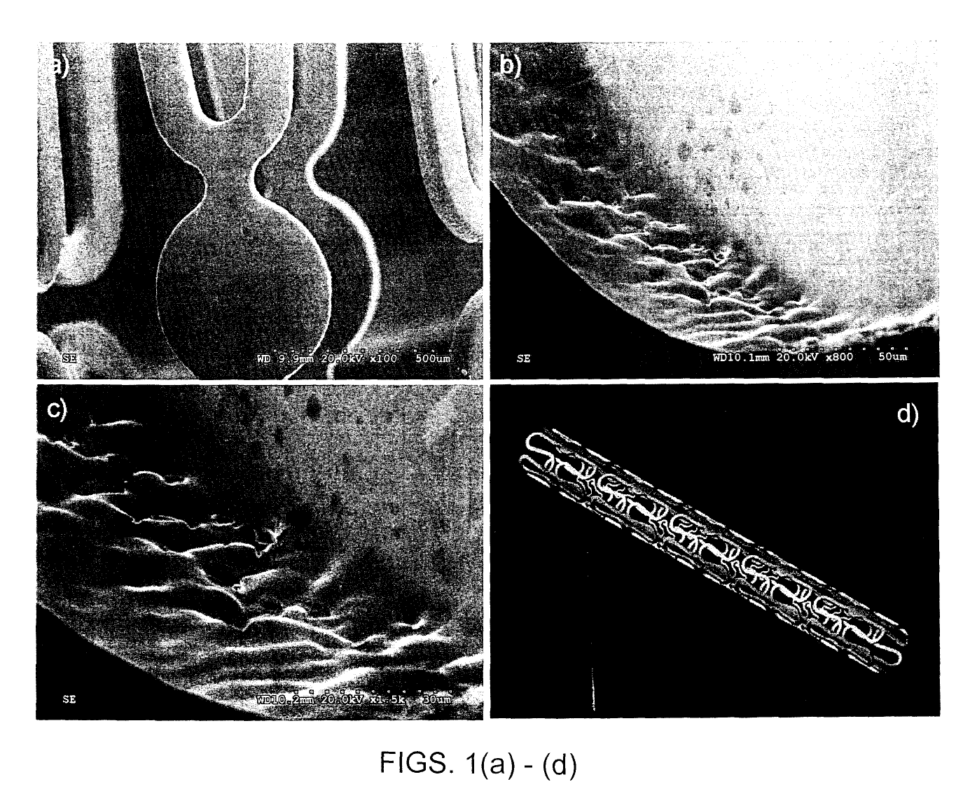

A 316L stainless steel stent, 14 mm in length and 0.85 mm outer radius was used. The stent surface was electro-polished, then cleaned in ultrasonic bath, with distilled water and then with ethyl alcohol. FIGS. 1(a) to (d) show the stent surface before coating. The radial surface non-uniformity is an artifact of laser cutting the stent from a steel tube during manufacture.

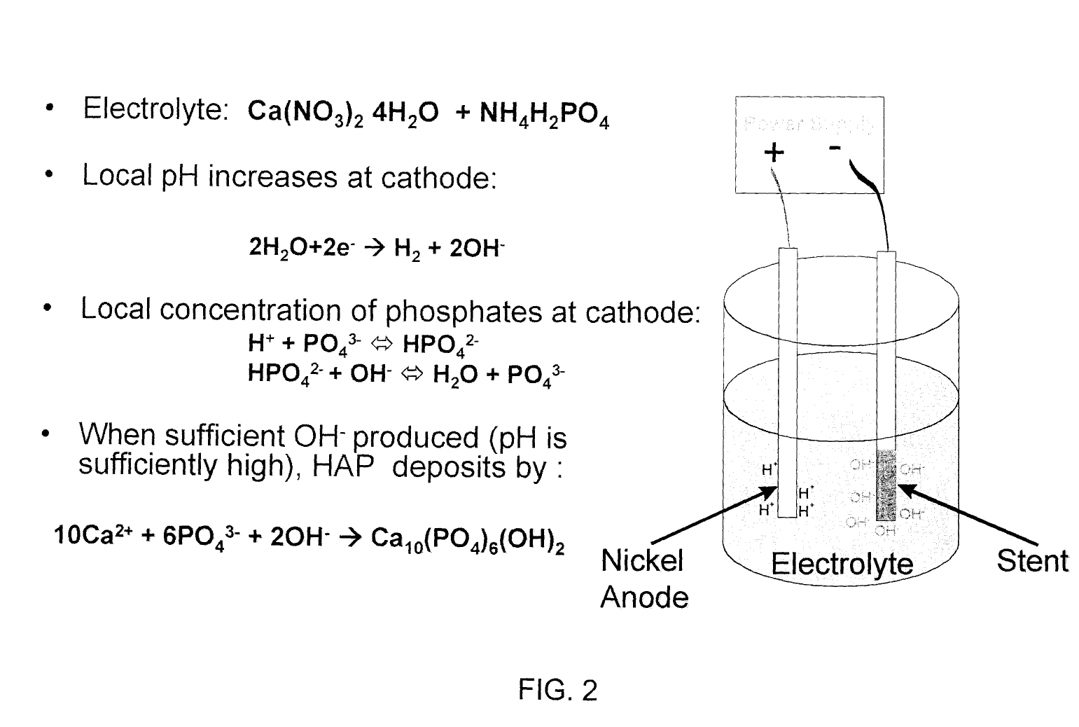

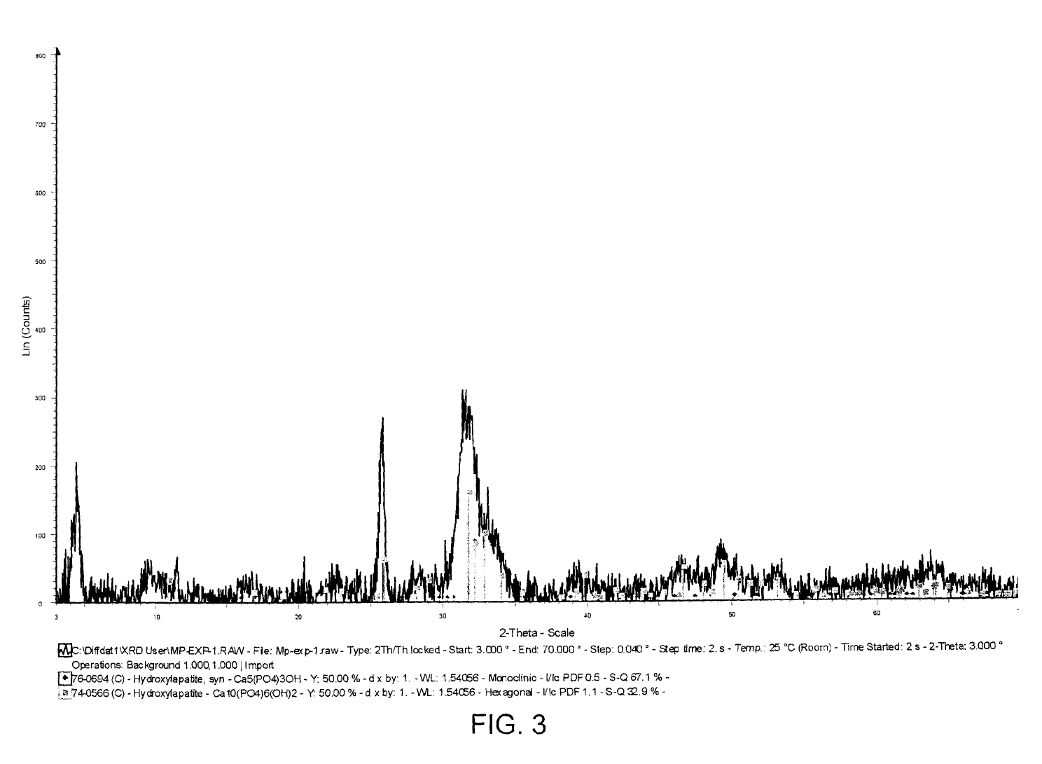

Electrochemical deposition was performed in 80 mL of electrolyte consisting of 0.02329M of Ca(NO₃)₂.4H₂O and 0.04347M of NH₄H₂PO₄, maintained at 50° C. The schematic ECD setup and relevant chemical reactions taking place during the process are shown in FIG. 2. The X-ray diffraction pattern from the material collected at the conclusion of ECD process is shown in FIG. 3, demonstrating the predominant presence of HAP in the material.

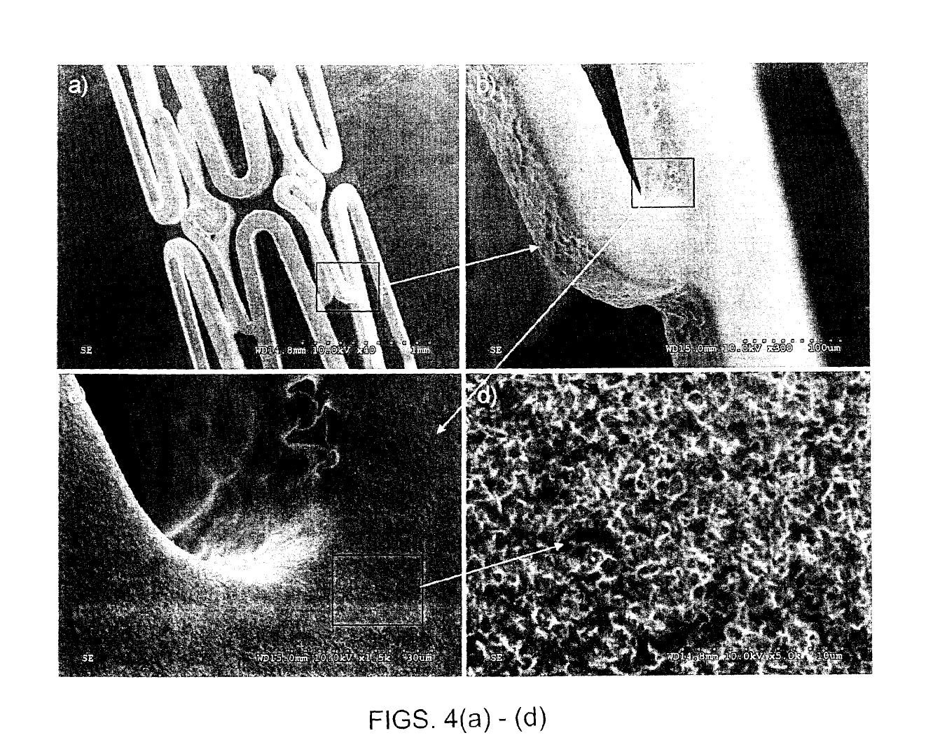

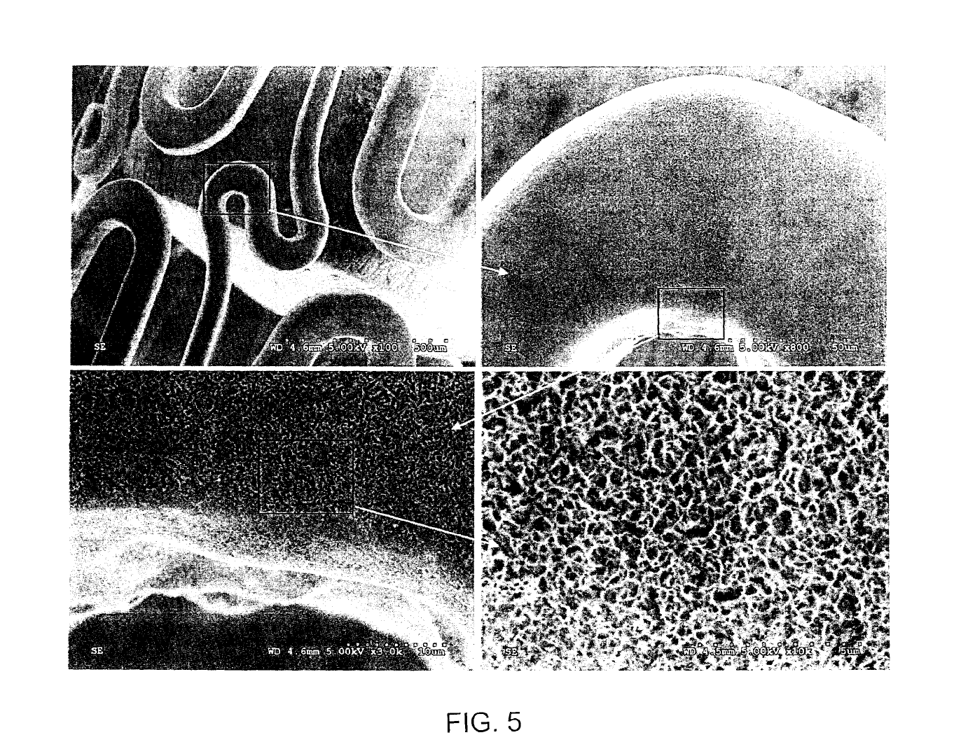

The “as-received” stent (i.e. without any additional surface preparation) was used as the cathode and a nickel ring was used as the anode. When a 0.90 mA current is applied for 50 seconds, an approximately 0.5 μm thin film HAP coating is deposited on the stent. Immediately, the coated stent was washed with running distilled water for 1 minute and air dried for 5 minutes. The coating had uniform coating approximately 0.5 μm thick that covered all surfaces of the stent, as shown in FIGS. 4(a) to (d). FIGS. 5(a) to (d) show the second sample processed in a separate repeat experiment.

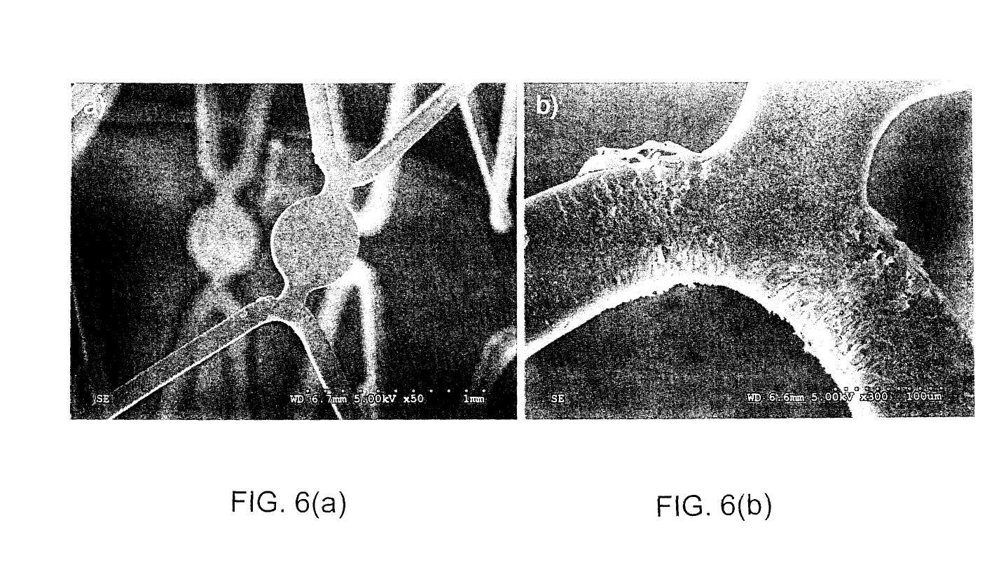

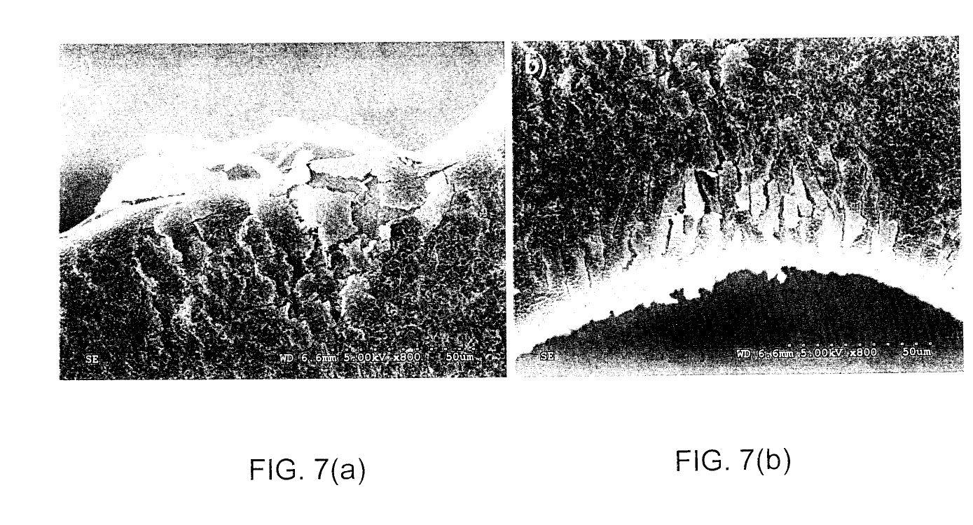

An expansion test was performed after the coated stent has been air dried. An Encore™ 26 Inflation Device Kit was used, and the catheter inserted into the stent was inflated to 170 psi. The expanded stent was observed under SEM. FIGS. 6 and 7 illustrate the results. The HAP coating separated from the stent surface in the areas of significant strain due to stent expansion. The flaked coating allowed assessment of the coating thickness, which was about 0.6 μm. The coating was retained on the stent in areas experiencing low strain or no strain.

### Example 2

### ECD on Pre-Oxidized Stent

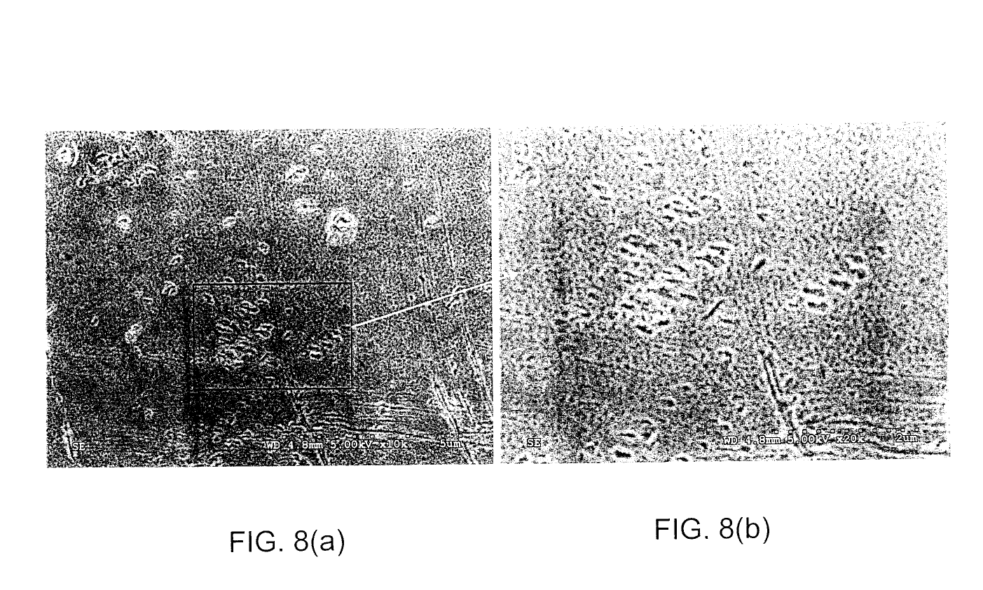

A 316L stainless steel stent was selected as in Example 1. The stent was cleaned in an ultrasonic bath, with distilled water and then with ethyl alcohol. Subsequently, the stent was subjected to an oxidation pretreatment to modify the surface for enhanced adhesion of coating. The stent was placed in a furnace at 500° C. for 20 minutes to create an oxidized surface layer on the stent. The oxide film thickness was estimated to be <50 nm thick. FIGS. 8(a) and (b) show the stent surface after oxidation pretreatment. The surface exhibits nano-roughness on the order of 50 to 100 nm.

Electrochemical deposition was performed as in Example 1, with immersion of the stent in 80 mL of electrolyte consisting of 0.02329M Ca(NO₃)₂4H₂O and 0.04347M NH₄H₂PO₄ at 50° C. The oxidation pretreated stent was used as the cathode and a nickel ring was used as the anode. When a 0.90 mA current was applied for 50 seconds, a thin film HAP coating was deposited on the stent. Immediately, the coated stent was washed with running distilled water for 1 minute and air dried for 5 minutes.

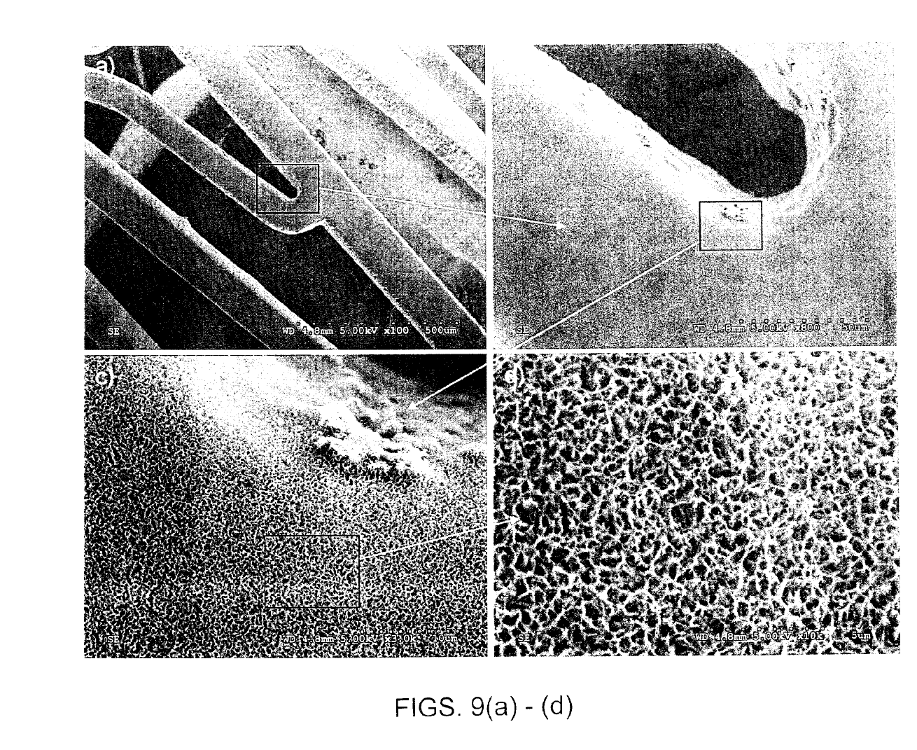

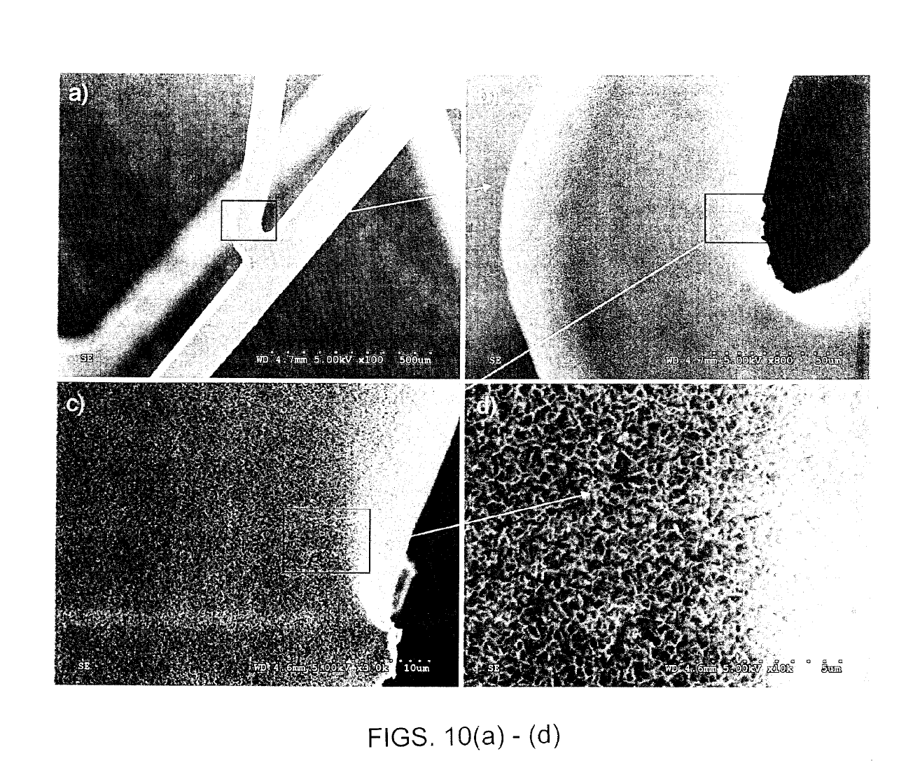

The coating uniformly covered the stent and the thickness was approximately 0.5 μm, as shown in FIGS. 9(a) to (d). The surface morphology of the coating remained unchanged from the HAP coating on the un-modified stent of Example 1 (FIGS. 4 and 5). An expansion test was performed as in Example 1 after HAP coated pre-oxidized stent had been air dried. As in Example 1, the expanded stent was observed under SEM, and FIGS. 10(a) to (d) illustrate the results. No separation of the coating was visible even in the areas of the highest strain due to the expansion, at magnifications up to 10,000×. The stent strain was accommodated by the coating through nano-size localized cracking, which was not visible under the microscope.

### Example 3

### ECD on Micro-Etched Stent

A 316L stainless steel stent was selected as in Example 1. The stent was cleaned in an ultrasonic bath, with distilled water and then with ethyl alcohol. In addition, the stent was subjected to a micro-etching pretreatment in an alkaline environment, followed by heat treatment in an oxidizing atmosphere. Alkaline treatment involved immersion in a 10N NaOH solution at 60° C. for 24 hours. The stent was then ultrasonically cleaned with distilled water and heat treated at 500° C. for 1 hour.

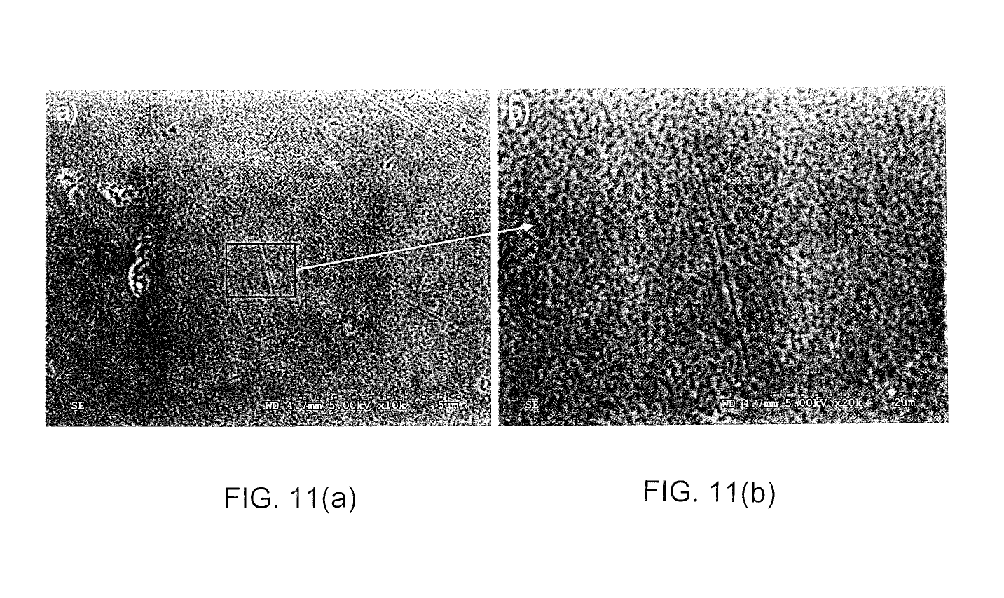

FIGS. 11(a) and (b) show the stent surface after alkaline micro-etching and oxidizing pretreatment. The surface exhibits nano-roughness on the order of 50 to 100 nm. Electrochemical deposition was performed done as in Example 1, with immersion of the stent in 80 mL of electrolyte consisting of 0.02329M Ca(NO₃)₂4H₂O and 0.04347M NH₄H₂PO₄ at 50° C. The alkaline pretreated stent was used as the cathode and a nickel ring was used as the anode.

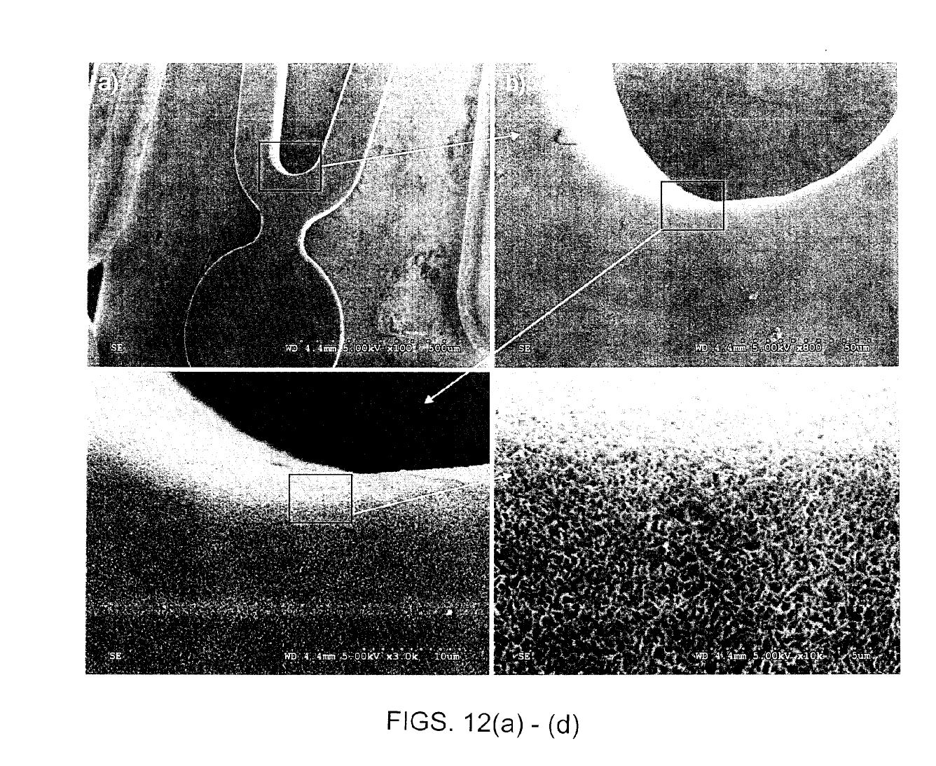

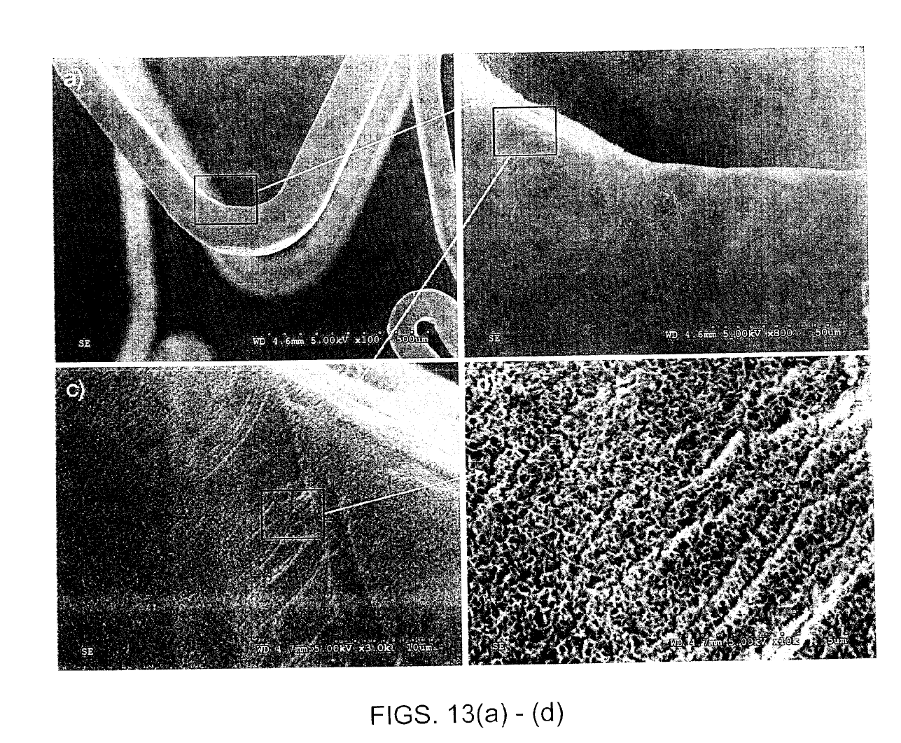

When a 0.90 mA current was applied for 50 seconds, a thin film HAP coating was deposited on the stent. Immediately, the coated stent was washed with running distilled water for 1 minute and air dried for 5 minutes. The coating uniformly covered the stent and the thickness was approximately 0.5 μm, as shown in FIGS. 12(a) to (d). The surface morphology of the coating remained unchanged from the HAP coating on the un-modified stent of Example 1 (FIGS. 4 and 5). An expansion test was performed as in Example 1 after HAP coated pre-oxidized stent had been air dried. As in Example 1, the expanded stent was observed under SEM, and FIGS. 13(a) to (d) illustrate the results. No separation of the coating was visible even in the areas of the highest strain due to the expansion. The plastic shear of the metal surface produced surface relief on the coating, without damage to the coating integrity. The stent strain was accommodated by the coating through nano-size localized cracking. In the highest strain areas, the nano-cracks linked to form 1 to 5 μm long microcracks; however, these microcracks did not trigger separation of the coating from the substrate.

### Example 4

### Reproducibility of ECD on Micro-Etched Stents

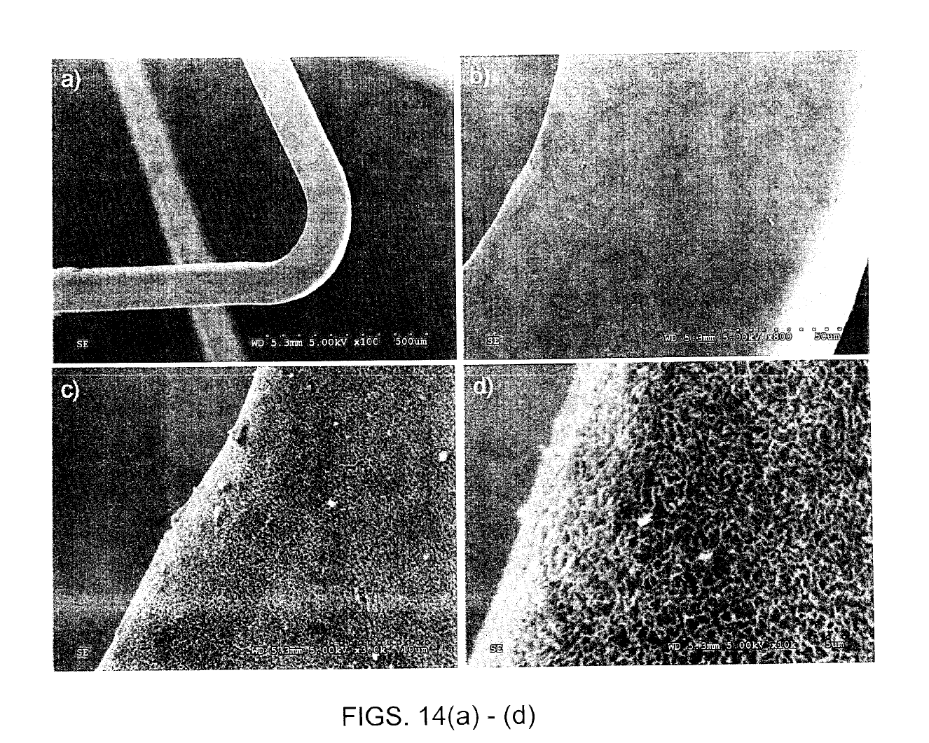

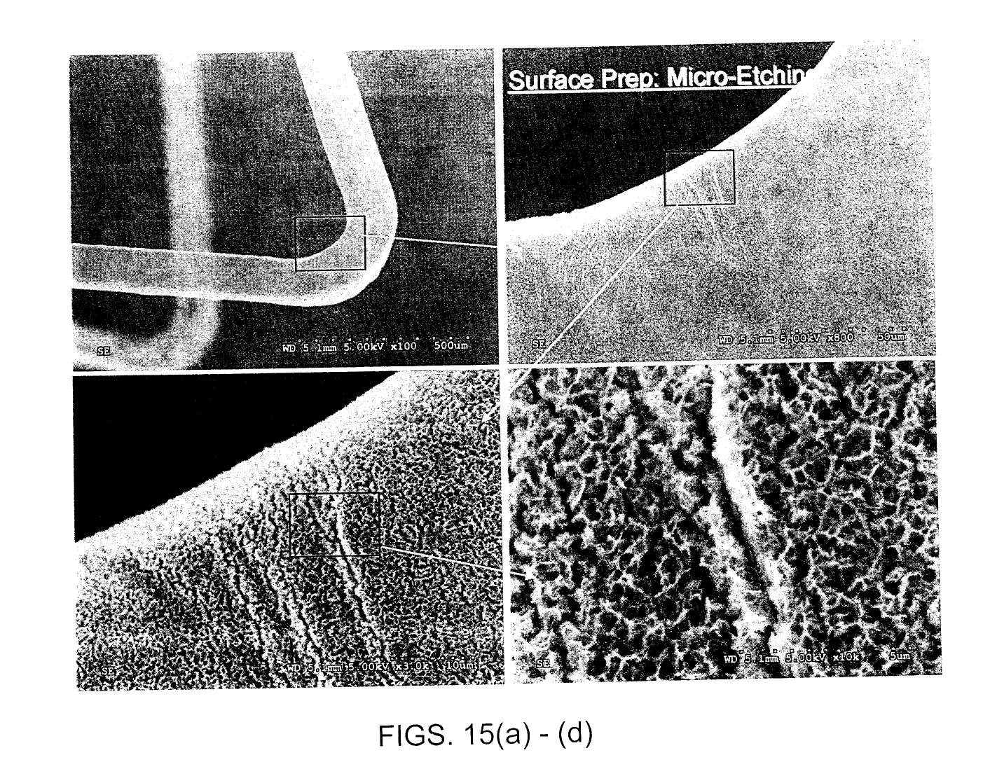

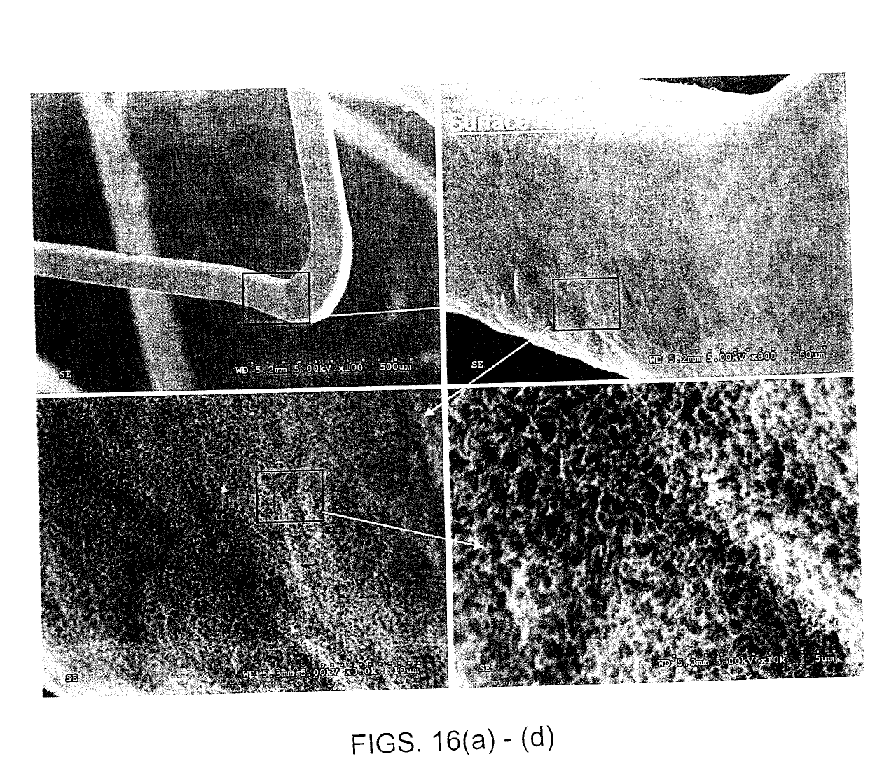

The experiment described in Example 3 was repeated exactly many times, and several randomly selected samples are presented here to verify process reproducibility. The expanded stents were observed under SEM, as shown in FIGS. 14 to 16. No separation of the coating was visible even in the areas of the highest strain due to the expansion. In one case (FIG. 15) microcracks appeared in the highest tensile strain area. Surface contamination of the coating was also visible, due to pick-up of foreign particles, likely during the drying of the coated stent. FIG. 16 illustrates extremely severe deformation of an over-expanded stent, including bending and twisting. However, even in this case, no damage of the coating was observed at magnifications up to 10,000×.

### Example 5

### ECD Deposition Time Effects

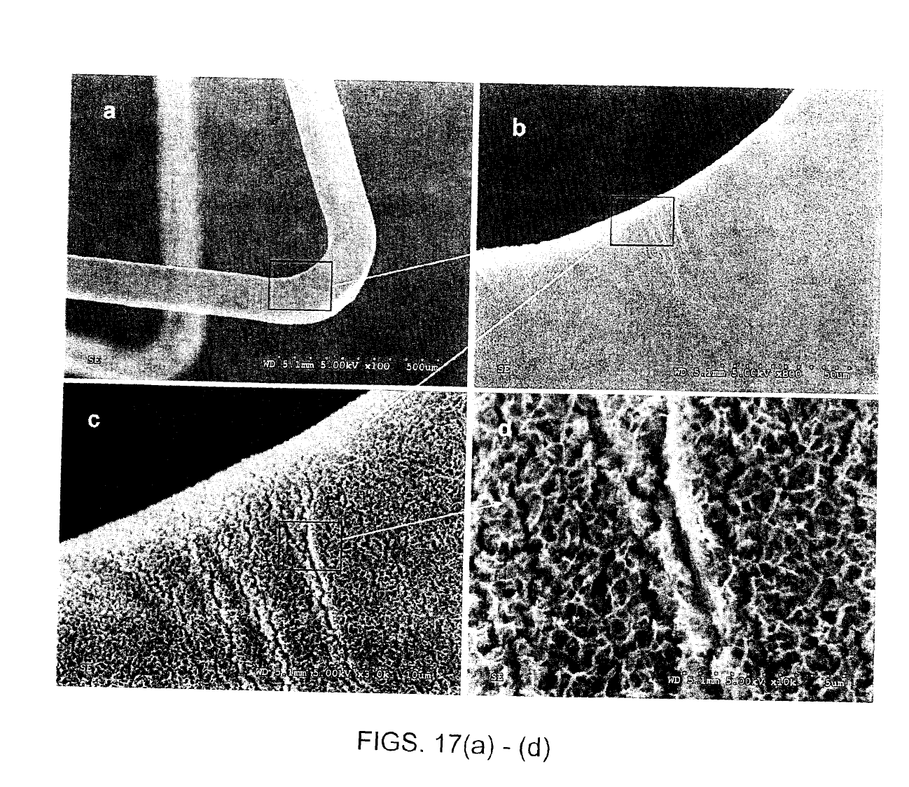

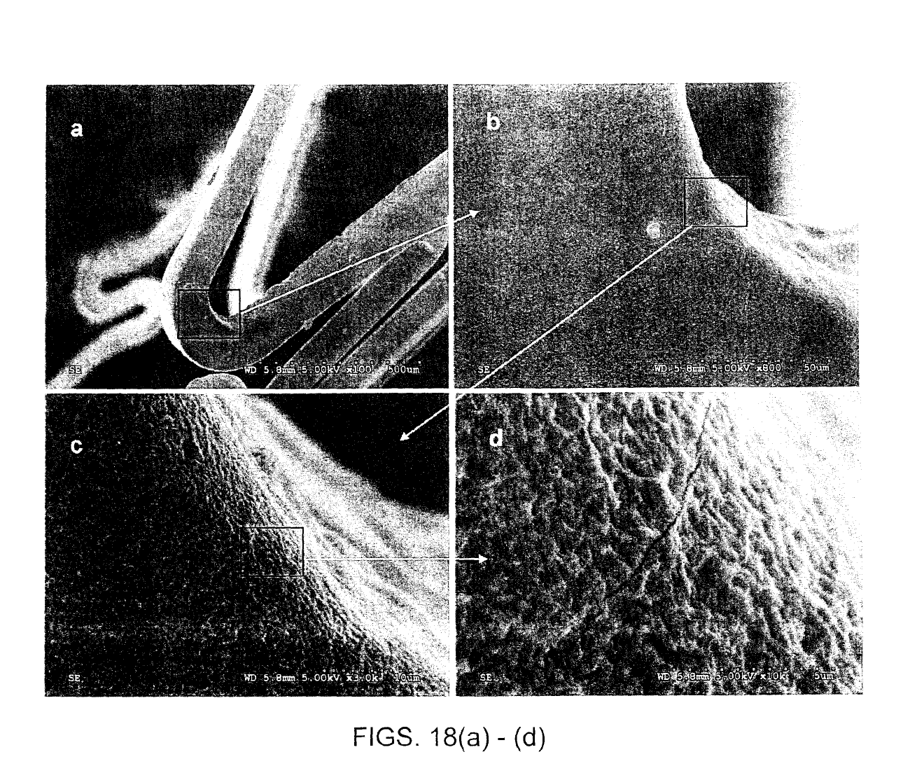

As in Example 1, three 316L stainless steel stents were electro-polished, and cleaned in an ultrasonic bath, first with distilled water and then with ethyl alcohol. As in Example 3, each stent was subjected to an alkaline micro-etch pretreatment. Electrochemical deposition was performed as in Example 1. A 0.90 mA current was applied for 60, 70 and 90 seconds to stents #1, #2 and #3 respectively, and a thin film HAP coating was deposited on the stents. The coating uniformly covered the stents and the thickness of the coating on stents #1, #2 and #3 was approximately 0.75 μm, approximately 0.90 μm and approximately 1.0 μm, respectively, as shown in FIGS. 17, 18 and 19 respectively. Some damage of the coatings starts to appear for the 60 second process (FIG. 17), although no separation of the coating from the substrate was observed. Longer processes (70 and 90 seconds) resulted in denser and thicker coatings, which were also stiffer and therefore produced larger stresses (at same strain) during stent expansion, as compared to the shorter deposition time/thinner/more porous coatings (FIGS. 13 to 17). It is therefore noted that the longer deposition times produce coatings which are not suitable for deforming substrates such as stents, but may be applied to non-deforming substrates such as dental or hip implants.

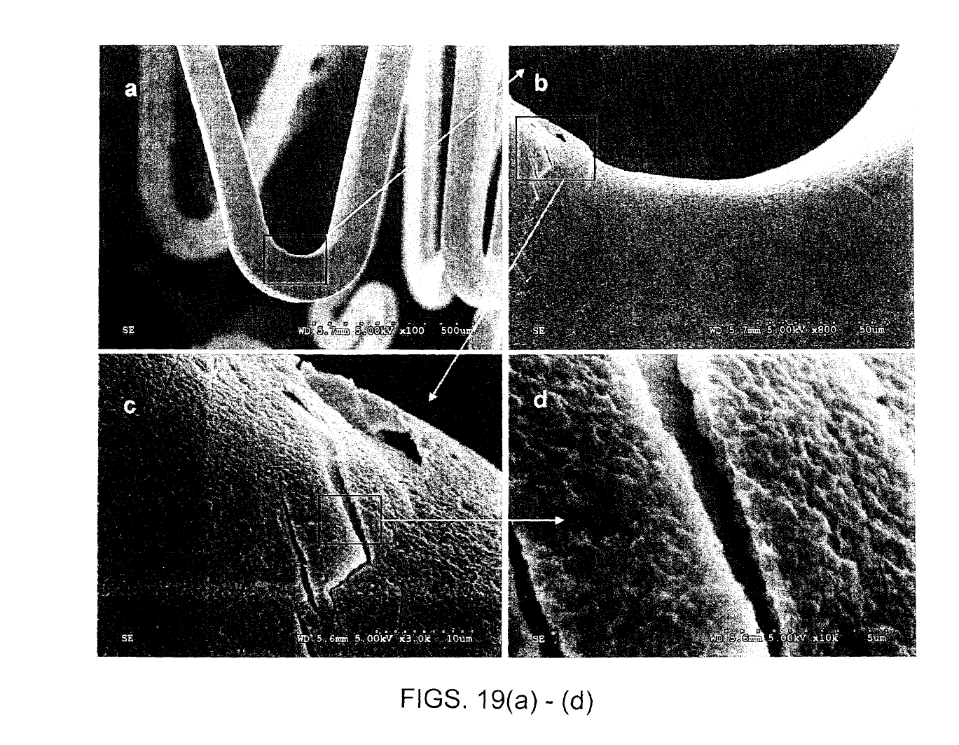

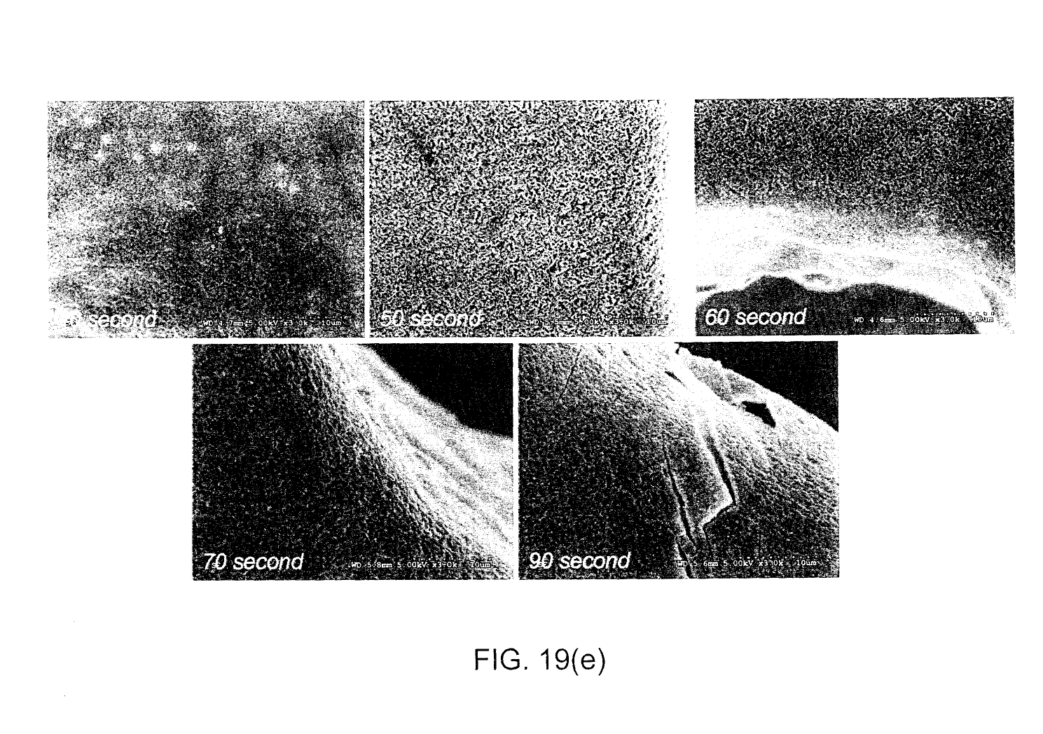

FIG. 19(e) comparatively illustrates the changing morphology of the HAP coated stents for different times of deposition. As deposition time increases, coating density increased and the coating was more uniform. 50 and 60 second depositions exhibited the most uniform coating structure, and the most desire porosity. 90 second deposition caused local delamination of the coating.

### Example 6

### ECD on Aero-Sol-Gel HAP Coated Stent

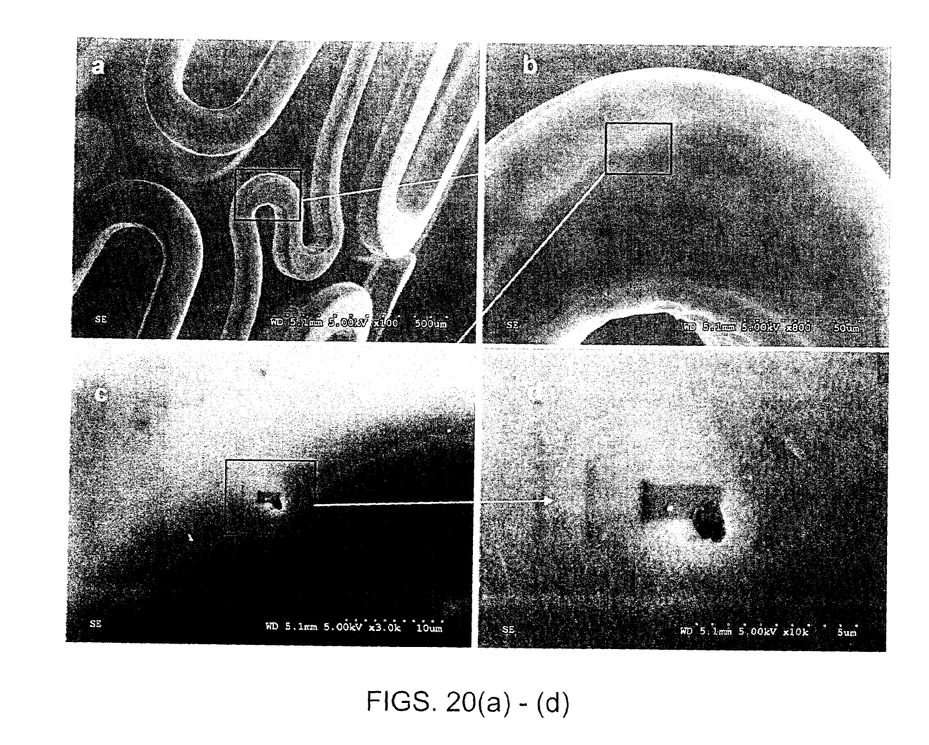

A 316L stainless steel stent was selected as in Example 1. The stent was cleaned in an ultrasonic bath, with distilled water and then with ethyl alcohol. The stent was coated with ˜0.2 μm thick coating of HAP using sol-gel technology as described in T. Troczynski and Dean-Mo Liu, “Novel Sol-Gel Hydroxyapatite Ceramic Coatings and Method of Making Same”, U.S. Pat. No. 6,426,114; Canadian Patent No. 2,345,552. The aero-sol-gel deposition (ASGD) method was used to uniformly deposit droplets of sol particles on the stent surface. A smooth, uniform, well-adhering film of HAP was produced on the stent surface as illustrated in FIGS. 20(a) to (d).

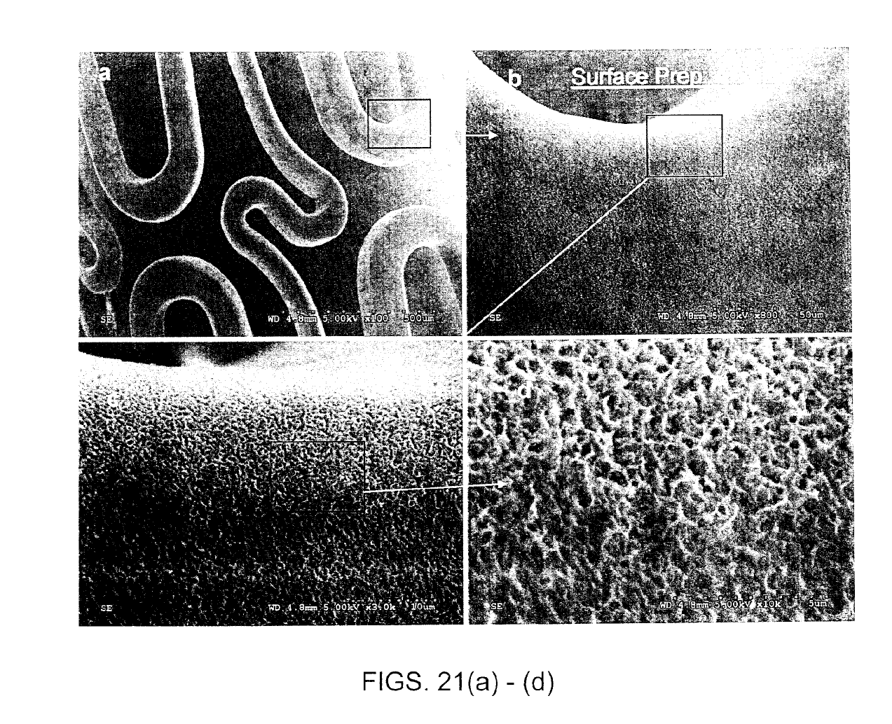

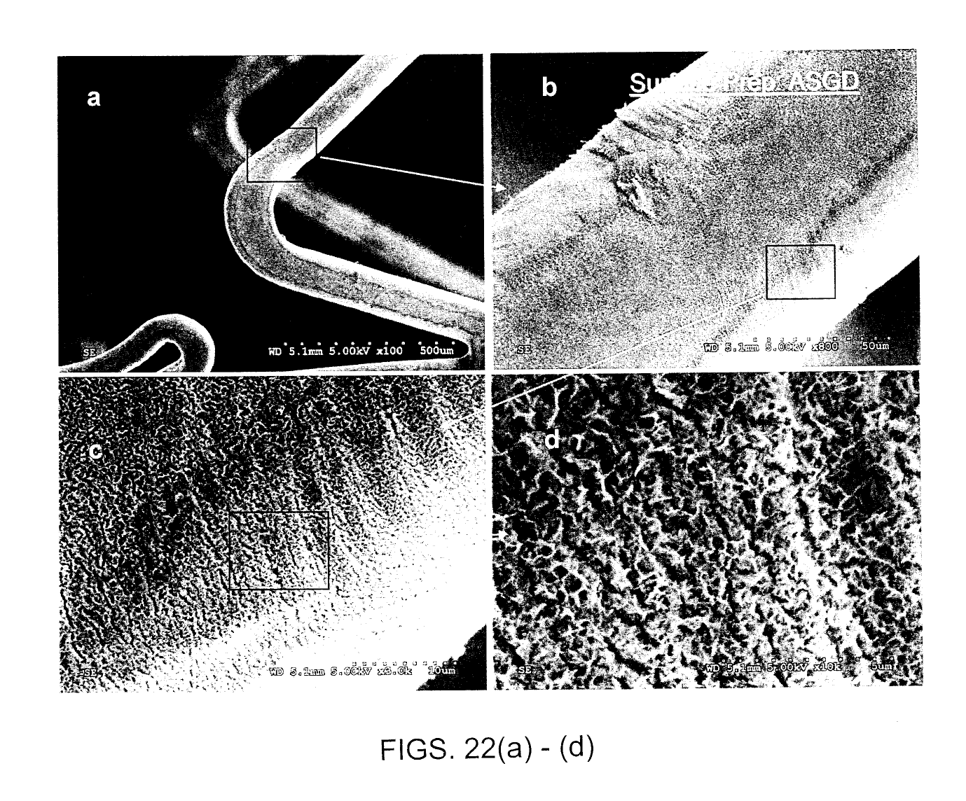

As this type (i.e., AGSD) of HAP film is very thin and adhesive, it survives the stent expansion test well. However, these types of very thin, dense films are not suitable for carrying additional materials such as bio-polymers, protein, and/or drugs. In this example we illustrate combination of the ASGD method with ECD method, in order to achieve a composite adherent film which is porous and thus capable to carry additional material such as bio-polymers, protein, and/or drugs. At the same time such ASGD-HAP pre-coated stent do not require any additional surface treatment (e.g. pre-oxidation or micro-etching) as the pre-existing film of ASGD-HAP has been found to provide a good nucleation surface for the precipitating HAP. This is illustrated in FIGS. 21(a) to (d) showing HAP deposited by ECD according to the process in Example 1, on an ASGD-HAP pre-coated stent, and FIGS. 22(a) to (d) illustrating the same stent after the expansion test performed according to Example 1. The surface morphology of the double-coated stent (FIGS. 21(a) to (d)) is the same as the directly-coated stent shown in Examples 1-4. The expanded double-coated stent (FIGS. 22(a) to (d)) shows dramatically improved performance of the coating (e.g. in terms of coating retention), as compared to the previously shown coatings on non-modified stents (FIGS. 7(a) to (b)). The stent strain was accommodated by the double coating through nano-size localized cracking. In the highest strain areas, the nano-cracks linked to form 1 to 5 μm long microcracks; however, these microcracks did not trigger separation of the coating from the substrate.

The ASGD-HAP nano-thin film provided sufficient surface modification to retain the subsequent ECD-HAP film on the stent during the expansion test. In this respect, the ASGD-HAP performs a similar function as stent pre-oxidation or micro-etching/oxidation as illustrated in Examples 2 and 3 respectively.

### Example 7

### ECD-HAP/PVA Bioceramic/Biopolymer Composite Coatings Through Co-Deposition

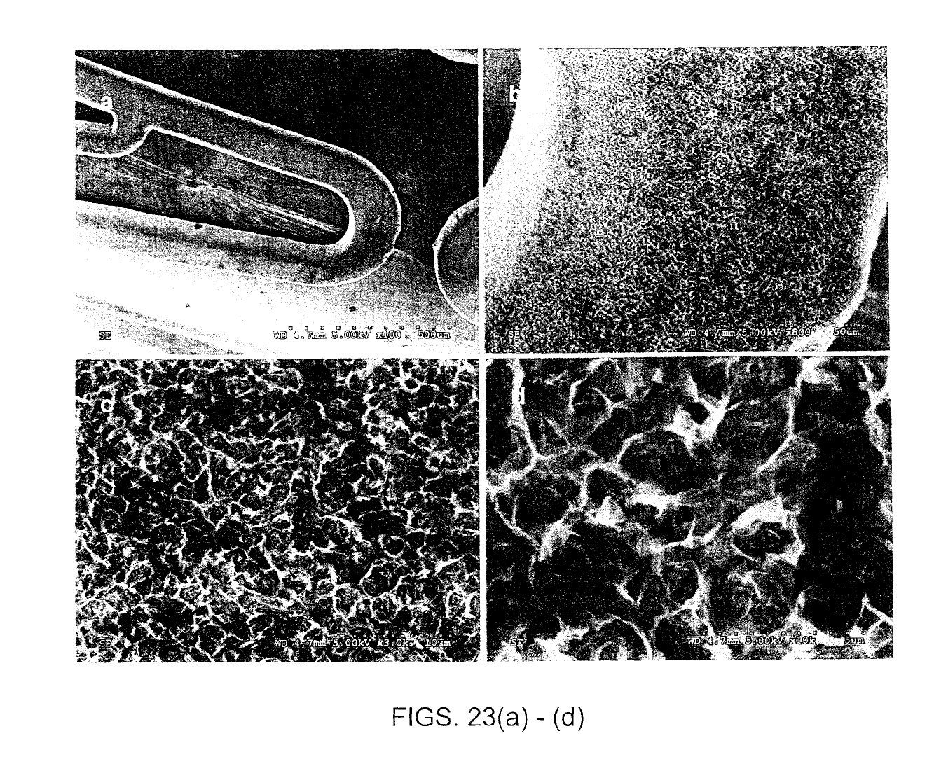

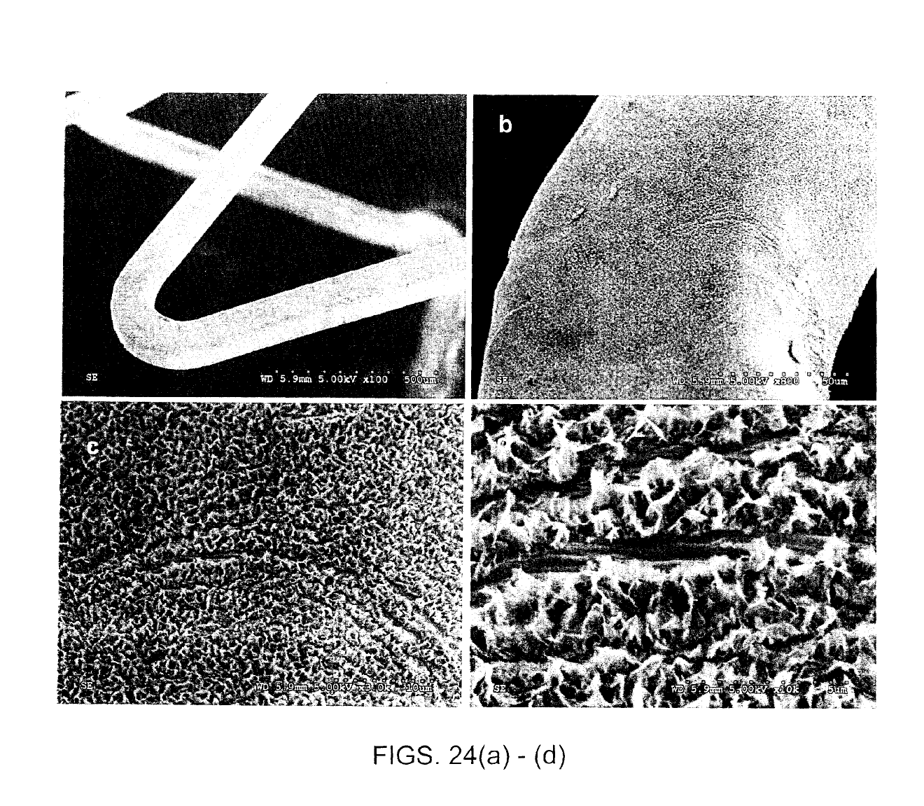

The principal advantages of the ECD process for HAP coatings (high porosity and room-temperature deposition) make them ideal carriers for organic materials such as polymers (preferably bio-polymers), proteins and DNA, and drugs. As an illustrative example of such composite coatings, the process described in Example 3 was modified by dissolving 0.1 g of polyvinyl alcohol (PVA) in the electrolyte (i.e. the coating solution, as described in Example 1), and the coating process was repeated as in Example 3. The resulting coating is shown in FIGS. 23 (a) to (d) (as-deposited) and FIGS. 24 (a) to (d) (after the expansion test).

The composite coating morphology appears more porous and more rough, as compared to the coatings produces without PVA (e.g. in Example 1). The expansion test exposes a fraction of the stent surface in the areas of highest strain (FIGS. 24(a) to (d)) but the coating was retained on the stent.

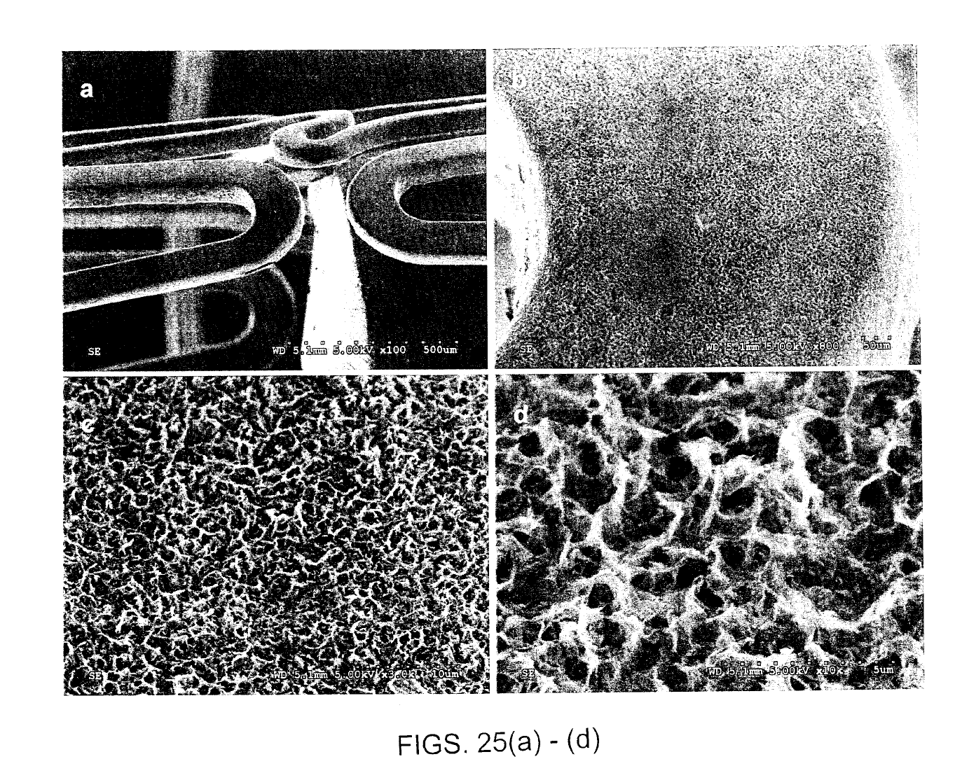

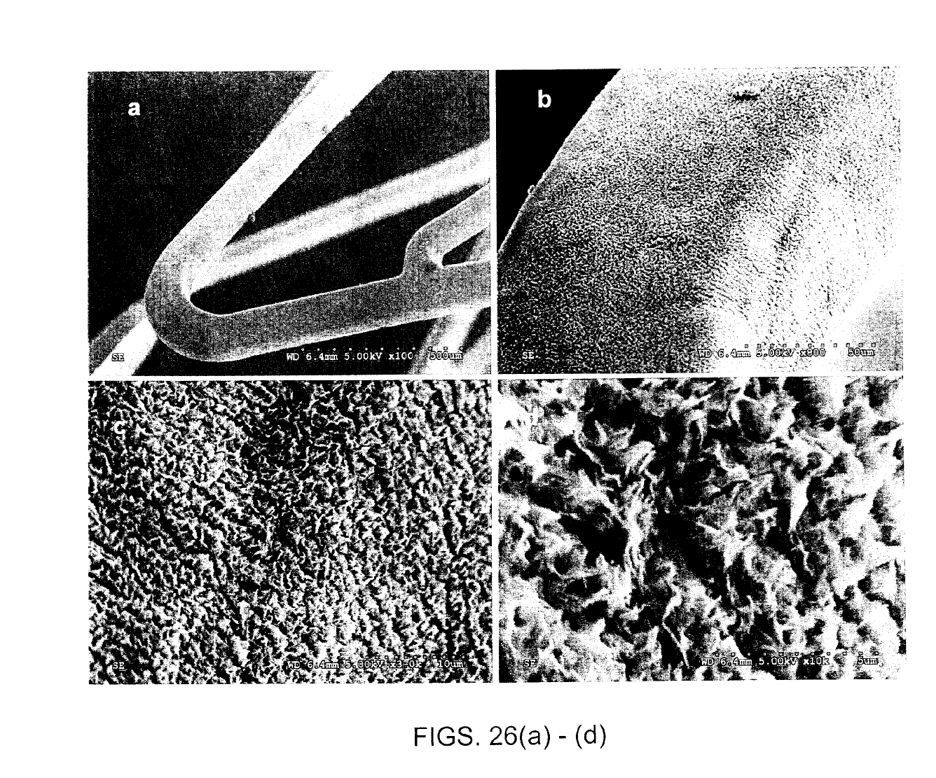

Increasing the amount of PVA dissolved in the bath to 0.5 g gave the results illustrated, before and after expansion, in FIGS. 25(a) to (d) and FIGS. 26(a) to (d), respectively. The surface morphology is more smooth (as compared to the 0.1 g PVA coating in FIGS. 23(a) to (d)), and the retention of the coating is also better. This may be attributed to the higher amount of PVA entrapped in the ECD-HAP coating, which helps to relax the expansion-related stresses through visco-elastic deformation (and thus stress relaxation).

### Example 8

### ECD-HAP/PLGA Bioceramic/Biopolymer Composite Coatings Through Impregnation

This example illustrates an alternate route to bioceramics/biopolymer composite coating, through post-deposition impregnation with a polymer solution. The experiment described in Example 3 is repeated. In addition, the HAP coated stent is impregnated with poly(DL-lactide-co-glycolide) (PLGA) solution of 2 wt % or 4 wt % PLGA and acetone, by simple dipping, followed by drying in air.

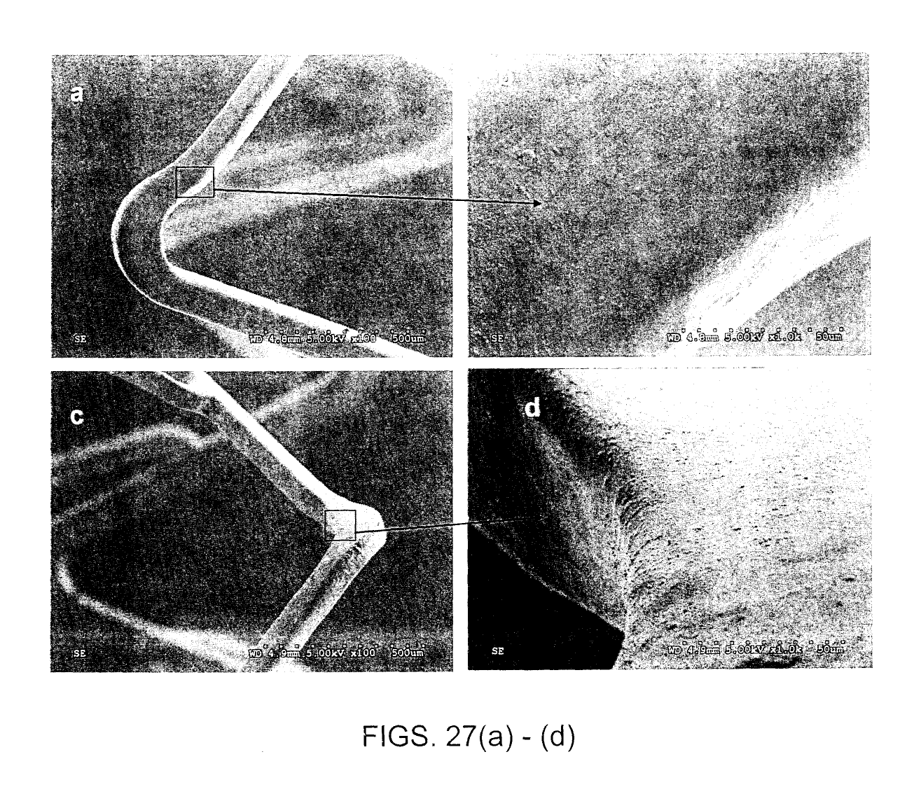

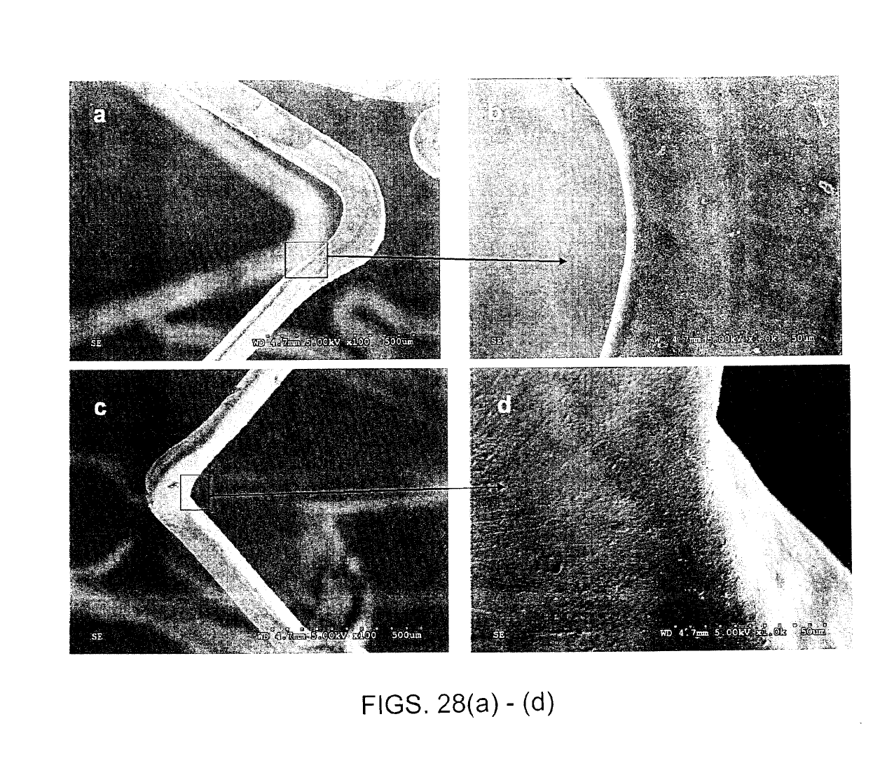

The over-expanded stent (i.e., strain up to two times greater than a normal implantation procedure) is illustrated in FIGS. 27(a) to (d) and 28(a) to (d) for the 2 wt % and 4 wt % PLGA solutions respectively. No separation of the coating is visible even in the areas of the highest strain due to the expansion in both cases, although a few microcracks may be observed for the coating impregnated with the 2 wt % solution of PLGA.

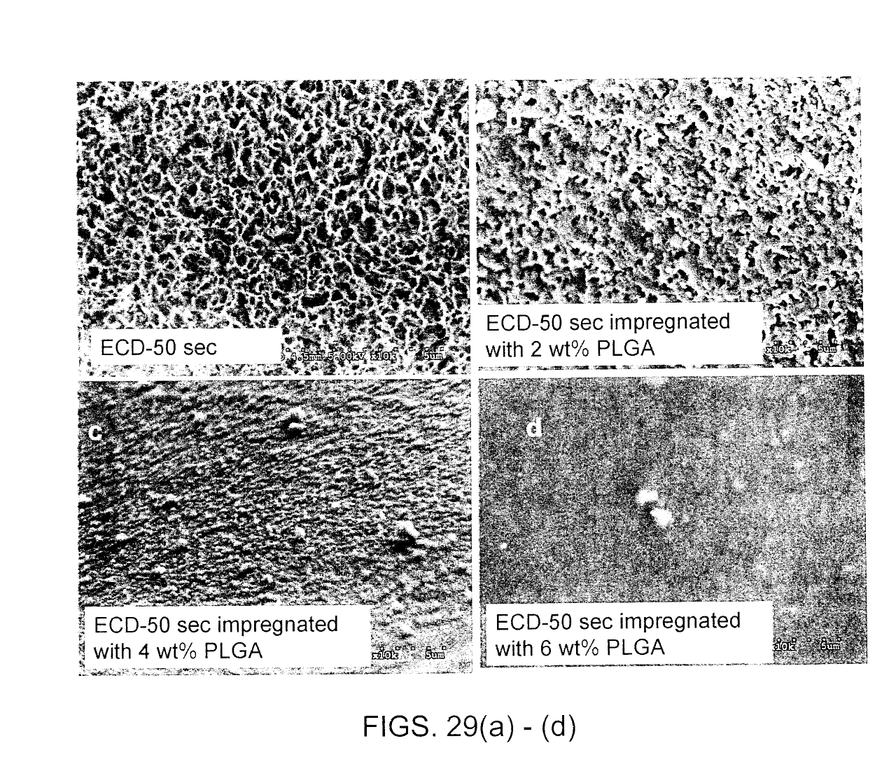

FIGS. 29(a) to (d) illustrate the surface morphology of the composite coatings, with an additional no-impregnation sample and a 6 wt % PLGA solution impregnation sample. FIGS. 29(a) to (d) show that PLGA filled in most of the pores of the HAP coatings with the 2 wt % PLGA solution, and PLGA filled in all pores of the HAP coating for 4 wt % PLGA solution; however, for these cases the surface features of the HAP coating can still be observed. The 6 wt % PLGA solution filled in all pores of the HAP coating and uniformly covered the surface of HAP coating to the extent that the features of HAP coating surface were no longer visible.

The illustrated bioceramics/biopolymer composite coatings have an important distinguishing feature. Namely, the ceramic (HAP) phase is a continuous phase, i.e. there is continuity between the ceramic, AND the polymer phase is also a continuous phase. The continuity of the ceramic phase makes the coating system stable and inhibits fast dissolution of the polymer phase into environment (e.g. tissue and body fluids). Such stable composite coatings are suitable for engineered drug delivery systems, for example.

What is claimed is:

1. A process of coating an implantable medical device with a calcium phosphate coating comprising:
(a) subjecting a substrate to a surface pretreatment whereby adhesion of the calcium phosphate coating to the substrate is enhanced;
(b) immersing the pretreated substrate in an electrolyte comprising calcium and phosphate species; and
(c) coating calcium phosphate onto the substrate by electrochemical deposition.

2. A process according to claim 1 wherein step (a) comprises heating the substrate to create an oxidized surface layer on the substrate.

3. A process according to claim 2 wherein the substrate is heated at 500° C. for 20 minutes.

4. A process according to claim 1 wherein step (a) comprises treating the substrate with an alkaline solution.

5. A process according to claim 4 wherein the alkaline solution is a sodium hydroxide solution.

6. A process according to claim 4 wherein treating the substrate with an alkaline solution comprises immersing the substrate in a 10N sodium hydroxide solution at 60° C. for 24 hours.

7. A process according to any of claims 4 to 6 wherein step (a) further comprises heating the substrate after the alkaline treatment step.

8. A process according to claim 7 wherein the substrate is heated at 500° C. for 1 hour.

9. A process according to claim 1 wherein step (a) comprises subjecting the substrate to aero-sol-gel deposition of hydroxyapatite.

10. A process according to claim 9 wherein the aero-sol-gel deposited hydroxyapatite has a thickness of approximately 0.2 μm.

11. A process according to any of claims 1 to 10 wherein the calcium phosphate coating has a thickness of less than or equal to approximately 1.0 μm.

12. A process according to claim 11 wherein the calcium phosphate coating has a thickness of less than or equal to approximately 0.75 μm.

13. A process according to claim 12 wherein the calcium phosphate coating has a thickness of less than or equal to approximately 0.5 μm.

14. A process according to any of claims 1 to 13 wherein the calcium phosphate coating is a coating selected from the group consisting of hydroxyapatite, dicalcium phosphate, tricalcium phosphate and tetracalcium phosphate.

15. A process according to any of claims 1 to 13 wherein the calcium species is Ca(NO₃)₂.4H₂O and the phosphate species is NH₄H₂PO₄.

16. A process according to any of claims 1 to 13 wherein the calcium phosphate coating is a coating of hydroxyapatite.

17. A process according to any of claims 1 to 16 wherein the electrolyte comprises an organic material.

18. A process according to claim 17 wherein the organic material is selected from the group consisting of a bioceramic, a biopolymer, a protein, a drug, DNA or combinations thereof.

19. A process according to claim 17 wherein the organic material is polyvinyl alcohol.

20. A process according to any of claims 1 to 19 comprising step (d) impregnating the calcium phosphate coated substrate with a polymer.

21. A process according to claim 20 wherein the polymer is a bioceramic or a biopolymer.

22. A process according to claim 20 wherein the polymer is poly(DL-lactide-co-glycolide).

23. A process according to any of claims 1 to 22 wherein the substrate is flexible.

24. A process according to any of claims 1 to 23 wherein the substrate is a stent.

25. A process according to claim 24 wherein the stent is a cardiovascular stent.

26. A process of coating an implantable medical device with a composite ceramic/polymer coating comprising:
(a) subjecting a substrate to a surface pretreatment whereby adhesion of the calcium phosphate coating to the substrate is enhanced;
(b) immersing the pretreated substrate in an electrolyte comprising calcium, phosphate and polymer species; and
(c) coating a continuous calcium phosphate phase and a continuous polymer phase onto the substrate by electrochemical deposition.

27. A process according to claim 26 wherein the calcium species is Ca(NO₃)₂.4H₂O, the phosphate species is NH₄H₂PO₄, and the polymer species is polyvinyl alcohol.

28. A process of coating an implantable medical device with a composite ceramic/polymer coating comprising:
(a) subjecting a substrate to a surface pretreatment whereby adhesion of the calcium phosphate coating to the substrate is enhanced;
(b) immersing the pretreated substrate in an electrolyte comprising calcium and phosphate species;
(c) coating a continuous calcium phosphate phase onto the substrate by electrochemical deposition; and
(d) impregnating the calcium phosphate coated substrate with a polymer to provide a continuous polymer phase.

29. A process according to claim 28 wherein the calcium species is Ca(NO₃)₂.4H₂O, the phosphate species is NH₄H₂PO₄, and the polymer is poly(DL-lactide-co-glycolide).

30. An implantable medical device comprising:
(a) a substrate with a surface micro-etched by alkaline pre-treatment; and
(b) a calcium phosphate coating deposited on the substrate by electrochemical deposition.

31. An implantable medical device according to claim 30 wherein the calcium phosphate coating has a thickness of less than or equal to approximately 1.0 μm.

32. An implantable medical device according to claim 31 wherein the calcium phosphate coating has a thickness of less than or equal to approximately 0.75 μm.

33. An implantable medical device according to claim 32 wherein the calcium phosphate coating has a thickness of less than or equal to approximately 0.5 μm.

34. An implantable medical device according to any of claims 30 to 33 wherein the calcium phosphate coating is a coating selected from the group consisting of hydroxyapatite, dicalcium phosphate, tricalcium phosphate and tetracalcium phosphate.

35. An implantable medical device according to any of claims 30 to 33 wherein the calcium phosphate coating is a coating of hydroxyapatite.

36. An implantable medical device according to any of claims 30 to 35 wherein the substrate is flexible.

37. An implantable medical device according to any of claims 30 to 36 wherein the substrate is a stent.

38. An implantable medical device according to claim 37 wherein the stent is a cardiovascular stent.

39. An implantable medical device comprising:
(a) a substrate with an oxidized surface layer; and
(b) a calcium phosphate coating deposited on the substrate by electrochemical deposition.

40. An implantable medical device according to claim 39 wherein the oxidized surface layer has a thickness less than or equal to 50 nm.

41. An implantable medical device according to claim 39 or 40 wherein the calcium phosphate coating has a thickness of less than or equal to approximately 1.0 μm.

42. An implantable medical device according to claim 41 wherein the calcium phosphate coating has a thickness of less than or equal to approximately 0.75 μm.

43. An implantable medical device according to claim 42 wherein the calcium phosphate coating has a thickness of less than or equal to approximately 0.5 μm.

44. An implantable medical device according to any of claims 39 to 43 wherein the calcium phosphate coating is a coating selected from the group consisting of hydroxyapatite, dicalcium phosphate, tricalcium phosphate and tetracalcium phosphate.

45. An implantable medical device according to any of claims 39 to 33 wherein the calcium phosphate coating is a coating of hydroxyapatite.

46. An implantable medical device according to any of claims 39 to 45 wherein the substrate is flexible.

47. An implantable medical device according to any of claims 39 to 46 wherein the substrate is a stent.

48. An implantable medical device according to claim 47 wherein the stent is a cardiovascular stent.

49. An implantable medical device comprising:
(a) a substrate with aero-sol-gel deposited hydroxyapatite layer; and
(b) a calcium phosphate coating deposited on the substrate by electrochemical deposition.

50. An implantable medical device according to claim 49 wherein the aero-sol-gel deposited hydroxyapatite layer has a thickness of approximately 0.2 μm.

51. An implantable medical device according to claim 49 or 50 wherein the calcium phosphate coating has a thickness of less than or equal to approximately 1.0 μm.

52. An implantable medical device according to claim 51 wherein the calcium phosphate coating has a thickness of less than or equal to approximately 0.75 μm.

53. An implantable medical device according to claim 52 wherein the calcium phosphate coating has a thickness of less than or equal to approximately 0.5 μm.

54. An implantable medical device according to any of claims 49 to 53 wherein the calcium phosphate coating is a coating selected from the group consisting of hydroxyapatite, dicalcium phosphate, tricalcium phosphate and tetracalcium phosphate.

55. An implantable medical device according to any of claims 49 to 53 wherein the calcium phosphate coating is a coating of hydroxyapatite.

56. An implantable medical device according to any of claims 49 to 55 wherein the substrate is flexible.

57. An implantable medical device according to any of claims 49 to 56 wherein the substrate is a stent.

58. An implantable medical device according to claim 57 wherein the stent is a cardiovascular stent.

59. An implantable medical device comprising a substrate with a composite coating, the composite coating comprising:
(a) a continuous ceramic phase; and
(b) a continuous polymer phase.

60. An implantable medical device according to claim 59 wherein the continuous ceramic phase comprises a calcium phosphate coating.

61. An implantable medical device according to claim 60 wherein the calcium phosphate coating is a coating selected from the group consisting of hydroxyapatite, dicalcium phosphate, tricalcium phosphate and tetracalcium phosphate.

62. An implantable medical device according claim 60 wherein the calcium phosphate coating is a coating of hydroxyapatite.

63. An implantable medical device according to any of claims 59 to 62 wherein the continuous polymer phase comprises an organic material selected from the group consisting of a bioceramic, a biopolymer, a protein, a drug, DNA or combinations thereof.

64. An implantable medical device according to any of claims 59 to 62 wherein the continuous polymer phase comprises polyvinyl alcohol.

65. An implantable medical device according to any of claims 59 to 62 wherein the continuous polymer phase comprises poly(DL-lactide-co-glycolide).

66. An implantable medical device according to any of claims 59 to 65 wherein the substrate is flexible.

67. An implantable medical device according to any of claims 59 to 65 wherein the substrate is a stent.

68. An implantable medical device according to claim 67 wherein the stent is a cardiovascular stent.

69. The use of a coated implantable medical device comprising a continuous ceramic phase and a continuous polymer phase in an engineered drug delivery system.
